(19) United States
(12) Patent Application Publication
(10) Pub. No.: US 2024/0307679 A1
(43) Pub. Date: Sep. 19, 2024

(54) USING POGO PINS FOR ELECTRO-STIMULATION

(71) Applicant: Ben Zion BEISKI, Kiryat Ono (IL)

(72) Inventor: Ben Zion BEISKI, Kiryat Ono (IL)

(21) Appl. No.: 18/437,145

(22) Filed: Feb. 8, 2024

Related U.S. Application Data
(60) Provisional application No. 63/445,741, filed on Feb. 15, 2023.

(51) Int. Cl. A61N 1/05; A61N 1/04; A61N 1/36

(52) U.S. Cl. CPC A61N 1/0551; A61N 1/0452; A61N 1/0476; A61N 1/36

(57) ABSTRACT

Electrostimulation finds diverse applications across medical, cosmetic, rehabilitation, sports, and lifestyle domains. Presently, stimulating probes commonly utilize fixed, self-adhesive patches, or metal-based pins connecting the tissue to the stimulation circuit. However, issues arise with the conventional patches, typically composed of Ag/AgCl or metal electrodes, typically, gold plating, as they often exhibit inadequate tissue contact, particularly over extended periods, and are susceptible to the effects of sweat or other body fluids. Additionally, the contact with the tissue is often interrupted in metal pins that are typically rigid and fail to adapt to the contours of tissues or changes in the contour resulting from movements such as running, walking, mechanical shocks, and vibrations. The inflexibility of these stimulating pins frequently leads to user discomfort, pain, and a loss of contact, diminishing the effectiveness of electrostimulation.

In contrast, pogo pins, equipped with embedded springs, naturally adjust the height of the electrostimulating pin to the contacting surface and tissue. This inherent adaptability makes pogo pins a markedly superior solution for electrostimulation applications, ensuring sustained tissue contact, stability during mevement, enduring contact reliability ever time, and enhanced user comfort.

Furthermore, electrodes based on pogo pins can be reused after cleaning, distinguishing them from most existing patches that are typically discarded after a singie use. This characteristic adds an ecofriendly dimension to the advantages of pogo-pin-based electrodes.

### CROSS-REFERENCE TO RELATED APPLICATION

None

### FIELD OF THE INVENTION

The current innovation primarily focuses on the electrostimulation of biological tissues through the utilization of electrostimulating electrodes featuring pogo pins.

### SUMMARY

The systems encompassed by the present invention comprise electrostimulator circuits that generate electrostimulation through electrical signals. These circuits are intricately linked to one or more electrostimulating electrodes, with a foundational design based on pogo pins. The electrostimulating unit is versatile, consisting of one or more pogo pins arranged in various configurations, and can be connected to one or more electrostimulator circuits, and/or a combination of pogo pins with other types of electrodes or patches.

In one aspect of the invention, a system featuring electrostimulating electrodes based on pogo pins is disclosed, with diverse applications in human health. Exemplary applications include but are not confined to, pain management, stimulation of salivary and/or lacrimal glands, implanted stimulator (like DBS), induction of bone growth, repair of damaged nerves, rehabilitation, physiotherapy, and management of pain associated with neurological and movement disorders, musculoskeletal disorders, metabolism, gastrointestinal tract disorders, GIT management, incontinence management, and more.

In another aspect of the invention, a system comprising electrostimulating electrodes based on pogo pins is presented, showcasing applications in the realm of cosmetics. These applications encompass eye lifting, anti-wrinkle treatments, facial care massage, eye and face massagers, skin smoothing, anti-aging solutions, skin tightening, skin lifting, skin rejuvenation, and similar cosmetic procedures.

In yet another aspect of the invention, a system with electrostimulating electrodes based on pogo pins is introduced, offering applications in lifestyle and sports. This extends to stimulating the abdominal area with belts, vests, or similar clothes, toner-training devices for muscles, fitness equipment, weight loss aids, and more.

Furthermore, the invention presents a system involving electrostimulating electrodes based on pogo pins, tailored for veterinary and animal treatment, particularly in the context of racing horses. An illustrative example is Functional Electrical Stimulation (FES), a subtype of NMES that selectively stimulates motor and/or sensory nerves. FES is employed to alleviate pain, and enhance muscle function, and is commonly used in the treatment of racing horses.

### BACKGROUND OF THE INVENTION

The present invention in electro-stimulation technology represents a significant advancement, enabling the creation of high-performance electrostimulation electrodes utilizing pogo pins. These electrodes find diverse applications in human health, spanning lifestyle, sports, and cosmetics.

Electro-stimulation, a replication of the natural processes orchestrated by the brain, serves various purposes. It seeks to induce muscular contractions, stimulate saliva secretion, and lacrimal gland secretion, activate sensory nerves, trigger local nerves and cells, and more.

Electrostimulation, encompassing modalities such as neuromuscular electrical stimulation (NMES), transcutaneous electrical nerve stimulation (TENS), and interferential therapy (IF), Functional Electrical Stimulation (FES), represents various forms of electrotherapy. This mode of treatment is acknowledged as a safe and effective approach with numerous health benefits, including pain relief and recovery for various conditions. Standard electrostimulation devices typically employ self-adhesive electrodes positioned around the targeted treatment area on the body. These electrodes are connected via wire leads to a control unit, facilitating the passage of electricity to interact with sensory and/or motor nerves and the surrounding tissue.

The present invention in electrostimulation technology represents a significant advancement, enabling the creation of high-performance electrostimulation electrodes utilizing pogo pins. These electrodes find diverse applications in human health, spanning lifestyle, sports, and cosmetics.

In a specific embodiment, employing a plurality of pogo pins as a contact point guarantees continuous contact with the skin/body, especially under significant changes in body contour or movement. This feature is particularly crucial for stimulators intended for prolonged use, typically spanning days, or for health applications such as pain management, saliva electrostimulation, urine stimulators, cardiac extracorporeal stimulators, skin rejuvenation, drug delivery, and similar therapeutic purposes.

Transcranial direct current stimulation (tDCS) is a form of neuromodulation that uses constant, low direct current delivered via electrodes on the head. It was originally developed to help patients with brain injuries or neuropsychiatric conditions such as major depressive disorder. It can be contrasted with cranial electrotherapy stimulation, which generally uses alternating current the same way, as well as transcranial magnetic stimulation.

In the realm of health, a Clinical Electrical Stimulation (CES) device transforms electricity into a tailored waveform for therapeutic purposes. The specific stimulation signals generated by this technology yield a range of effects, including promoting muscle contraction for strength enhancement, alleviating pain, managing preoperative and postoperative pain in patients with spinal injuries, improving blood circulation, facilitating physiotherapy and rehabilitation, treating dry eyes, aiding in stroke rehabilitation, increasing saliva production, managing incontinence, and supporting precision spinal cord stimulator systems such as Precision Montage MRI, Precision Spectra, improving the stimulating electrodes in implantable devices (DBS, defibrillator, pacemaker), and more.

Furthermore, electro-stimulation plays a pivotal role in Digital Nerve Assessment Technology, offering a solution to the challenges posed by electromyography (EMG) in intraoperative neuromonitoring. It is deployed during surgical procedures to assist in locating and mapping motor nerves through the utilization of mechanomyography (MMG) signals and electrical nerve stimulation. This information enables the assessment of a patient's neurophysiological status by measuring and comparing MMG signals throughout the surgical procedure.

In the realm of sports, electro-stimulation serves as a notable example of inducing muscular contractions. In this process, an electro-stimulator transmits electrical impulses to various muscle groups, prompting involuntary contractions. This method enhances the engagement of a greater number of muscle fibers during training, leading to an elevated intensity of effort compared to conventional training methods.

An increase in saliva secretion, often employed in the treatment of dry mouth, involves electrostimulation. The electrostimulator's signals elicit evoked potentials in the nerves associated with both the salivary glands and the salivatory center in the brain. These evoke potentials prompt the salivary glands to produce more saliva, mimicking the commands originating from the salivatory center in the brain through efferent fibers. Simultaneously, the signals conveyed to the salivatory center via afferent fibers communicate the presence of foreign objects, such as food, in the oral cavity. The brain processes this sensory information, interpreting it as a directive to instruct the salivary glands to increase saliva secretion—a phenomenon akin to the findings demonstrated by Pavlov in his renowned experiments. Noteworthy nerves involved in this process include the lingual nerve, linking the sublingual and submandibular glands to the brain, and the auriculotemporal nerve, connecting the parotid glands to the brain.

Cosmetic electrotherapy employs low electric currents transmitted through the skin to elicit a range of therapeutic effects. These include muscle toning throughout the body and subtle facial enhancements like eye lifting. This technique encompasses various treatments such as anti-wrinkle instruments, facial care massage, eye-face massagers, skin smoothing, anti-aging procedures, skin tightening, skin lifting, skin rejuvenation, and more.

Whether undergoing recovery from an injury, or stroke or managing the challenges of conditions like fibromyalgia, patients may find substantial benefits in procedures incorporating electrical stimulation.

Various wearable electro-stimulators, including those based on pogo pins, textile-based models, knitted integrated electro-stimulators, and fashionable planar circuit boards, find application in diverse electro-stimulation scenarios integrating an array of pogo pins into a wearable suit, such as on the upper torso providing the advantage of electro-stimulation even during motion without causing tissue damage or compromising contact.

The versatility of pogo pins is underscored by their availability in different lengths, inner structures, current specifications, and stroke movements (depicted in FIG. 3d, showcasing the displacement of the pogo pins edge due to spring compression). This diverse range facilitates the selection of the most suitable pogo pins based on specific requirements and the location of the body where the wearable device is positioned.

An implanted electro stimulator such as a deep brain stimulator (DBS), defibrillator, pacemaker, and the like, where the electrodes (pogo pins contact) the tissue and adjust themselves to the changes in the tissue, such as due to the heartbeats. Due to its unique structure, electrodes can now be placed at other locations in the heart, where the movement of the tissue inhibits placing fixed-tip electrodes.

### Pogo Pins

Pogo pins (100), also referred to as a spring-loaded pin, is an electrical connector mechanism characterized by an integrated helical spring within the pin. This design ensures a constant force is exerted against the back of the mating receptacle, typically a Printed Circuit Board (PCB). This counteraction effectively prevents any undesired movement that could potentially result in intermittent connections, avoiding interruptions in stimulation and the generation of unintended signal patterns.

The configuration of pogo pins comprising of three fundamental components: the barrel (121), the spring (122), and the plunger (120).

When external force is applied to the pin, the spring undergoes compression, causing the plunger to move within the barrel. The specific design of the barrel serves to secure the plunger, preventing the spring from expelling it when the pin is not in a locked position. Pogo pins can be structured with various internal configurations, as depicted in FIG. 9 and FIG. 6. These include the back drill (129), bias tail (130), ball-based (131), cap (132), right angle (where the through-hole pin is at a 90-degree angle to the pogo pins) (124), double-ended (where both sides are connected to the spring) (123), a waterproof version suitable for wet environments, and an insulating version wherein the pogo pins(s) are encased in a thermoplastic enclosure.

A pogo pin requires a slight clearance between the plunger and the barrel to facilitate smooth sliding, although momentary disconnections may occur due to vibrations or movement. To address this issue, the plunger is typically designed with a slight tilt to ensure a continuous connection. This may involve the addition of a ball between the two components, or the plunger may feature an angled or countersunk tip.

Materials used in pogo pins—The plunger and barrel of pogo pins commonly utilize brass (152) or copper as the foundational material, with a thin layer of nickel (151) applied. The final finishing touch often involves a layer of gold (150), a biocompatible material that enhances inherent durability and contact resistance. The springs (122) are typically crafted from copper alloys or spring steel or other non-magnetic materials like Nitinol, aluminum, and the like.

In the realm of bioelectronic interfaces, electrodes must exhibit mechanical flexibility and chemical inertness. While skin and tissue contact are pivotal in most electrostimulator pins, chemical inertness ensures that electrodes do not react with biological fluids or living tissues. Pogo pins, typically coated with a layer of gold plating (e.g., gold (150)) or other conducting material like silver, platinum, conducting polymer (poly 3-alkylthiophenes, polyacetylene, polypyrrole, polyindole and polyaniline and their copolymers) are designed with inherent coating/plating that imparts chemical inertness. The unique spring mechanism of the pogo pins ensures uninterrupted contact between the skin/body and the electrodes, even during movements or changes in body contour.

Pogo pin tips refer to the components of the pogo pins that come into contact with tissues through their edges or tips. These pins offer a diverse range of tipping edges, allowing for the selection of specific tips tailored to particular applications. In FIG. 5a, a variety of optional tips for pogo pins is illustrated, including a flat head (200), a concave tip (201) suitable for the application of gel, if necessary, a small pins-head (202), a pointing edge (203) capable of pinching and penetrating the skin to a desired depth, and a small area flat head (204).

The selection of pogo pin heads offers the versatility to create an electro-stimulator that can either penetrate the Stratum Corneum (203), make direct skin contact with a flat head (200), or employ any combination thereof. This adaptability allows for precise optimization of contact, enhancement of the signal-to-noise ratio, and mitigation of the impact of motion artifacts on the signals.

Pogo pins and resistance to lateral forces—Pogo pins are engineered to withstand lateral forces without fracturing. These forces may be exerted during the insertion of the electro-stimulator pin into its designated position, such as when placing a saliva stimulator in the oral cavity or positioning a mask with an array of electro-stimulators for skin rejuvenation. Additionally, the pogo-pin structure (see FIG. 5b) ensures resilience against substantial lateral forces.

Waterproof pogo pins—Waterproof pogo pins (133) are designed to function effectively in environments with moisture, sweat, high humidity, implanted inside organs, and similar conditions.

Non-magnetic pogo pins—Pogo pins can be manufactured with non-magnetic material allowing its use in strong magnetic environments such as Magnetic Resonance Imaging (MRI). The materials to be used can be, Brass, Aluminum, Nitinol, Polymers with spring properties such as helical carbon-phenolic-based polymer, Polyacetal (POM) springs made from the thermoplastic polymer polyoxymethylene, and the like.

### BRIEF DESCRIPTION OF THE DRAWINGS

The present invention will be described in greater detail in the following description of the invention with reference to the accompanying drawings that form a part hereof and are given merely as examples of different embodiments and configurations of the present invention, in which:

FIG. 1 shows the flow of the electro-stimulating signal from the electro-stimulating signal generator 140 to the pogo pins 100, through the barrel 121 to the plunger 120, the spring 122, and to the tissue 170. It is controlled by an embedded or remote-control module 316 that can get inputs from a biofeedback 317.

FIG. 2 illustrates a setup of patch-based 161 electro-stimulator electrodes based on pogo pins 100 and a standard connector (the type in use in ECG patches) 162.

FIG. 3a shows Surface Mounted Device (SMD) pogo pins with a soldering flat surface 126.

FIG. 3b shows a pogo pins with a through-hole pin 125.

FIG. 3c shows the different variations in the pogo pins' heights and strokes 127.

FIG. 3d shows the typical variables in the dimensions of pogo pins.

FIG. 4 shows a setup in which a number of pogo pins 100 are connected to a PCB 165 and receive the same stimulating signals from the electro-stimulation generator 140. The signal is transferred via each one of the pogo pins 100 to tissue 170. The number of pogo pins 100 connected to a single signal generator can vary and is not limited.

FIG. 5a illustrates the different types of pogo pins' tips (edge), Flat 200, concaved 201 multiple spikes 202, pointed edge 203, and small flat head 204.

FIG. 5b shows how a pogo pin can withstand lateral forces 160 here in a slip-ring example.

FIGS. 5C-1 to 5C-5 show various heads of pogo pins with optional dimensions and shapes.

FIG. 6 shows a typical setup of pogo pins in a connector. The insulating housing 111, the piston 120, the body (or the barrel) 121, the inner (embedded) spring 122, and the tail (through hole pin) 125. FIG. 2b shows pogo pins with an SMD contact 126 and a rolling ball 128.

FIG. 7 illustrates a potential setup of an array of pogo pins that function as one electro-stimulator electrode over a patch 161. It can be, as an example arranged in an arc like 163 or a rectangular array 164.

FIG. 8 shows the typical metallurgic structure of a pogo pins. A brass base 152, a thin coating of an intermediate layer of nickel 151, and an outer coating of gold 150.

FIG. 9 is a schematic view of the different types of pogo pins. Back drill type 129, bias tail 130, ball 131, cap 132, waterproof 133, right angle 124, double-ended 123, and waterproof 133.

FIG. 10 shows different types of pogo pins 100 (in height, stroke length, type, etc.) that can be combined into a single stimulating electrode. This degree of freedom allows even challenging tissues and tissue contours. It can be connected to a rigid PCB 165. The stimulated tissue is 170, and the stimulating signal generator 140.

FIG. 11 shows a setup in which a number of pogo pins 100 are connected to a flexible PCB 166 and receive the same stimulating signals from the electro-stimulation generator 140. The signal is transferred via each one of the pogo pins 100 to the tissue 170. The number of pogo pins 100 connected to a single signal generator can vary and is not limited.

FIG. 12 shows a setup of electro-stimulator electrodes for increasing saliva secretion using pogo pins 100 as electrodes. Two elastic arms 106 are inserted into the oral cavity. The stimulating signal passes from the electronic-control modules 108 from the stimulating signal generator 140 and via the connector 105 via wires 103 that are coated with an insulating silicon layer 102 to the plate 107 and the stimulating electrode 100, acting as a pair for positive and negative stimulating signals.

FIG. 13 shows a setup of electro-stimulator electrodes for increasing saliva secretion using pogo pins 100 as electrodes placed on a plate 107 that is connected to an ear plug, that serves as an anchoring point, and flexible arms 108 connected to a control unit.

FIG. 14 shows a setup of electro-stimulator electrodes for increasing saliva secretion using pogo pins 100 as electrodes placed on a plate 107 that is connected to an earplug 109, that serves as an anchoring point, and flexible arms 108 connected to a control unit.

FIG. 15 shows a setup of electro-stimulation with pogo pins embedded into the stimulation patches 301 within a fabric, located on the abdomen and at various places on the body 300.

FIG. 16 shows a setup of electro-stimulation with pogo pins embedded into the stimulation patches 303, for treating horses and located in the various places on the horse's body.

FIG. 17 shows a location for the setup of electro-stimulation with pogo pins embedded for stimulating the parotid glands 304, the neck 305, the sub-lingual glands 308, the throat 307 the sub-mandibular glands 306.

FIG. 18 shows a setup of electro-stimulation with pogo pins embedded into a head mask for stimulating the cranial 309.

FIG. 19 shows the schematic structure of a Deep Brain Stimulator (DBS) where the implanted electrode has pogo pins at the tip 310 and it receives the stimulating signal from an implanted stimulating pulse generator 311.

FIG. 20 shows the schematic of the implanted cardioverter defibrillator with the stimulating pulse generator 311 and the stimulating electrodes with pogo pins tip 312.

FIG. 21 shows the pacemaker electrodes, with pogo-pin tips, placed at the heart's right atrium 314, coronary sinus 313, and the right ventricle 315.

### DETAILED DESCRIPTION OF THE INVENTION

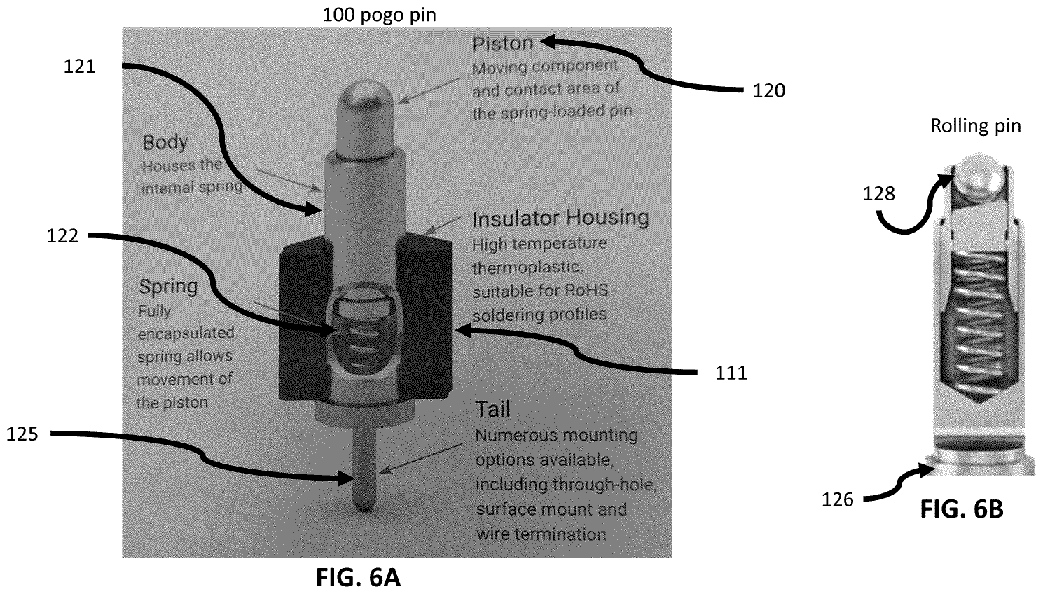

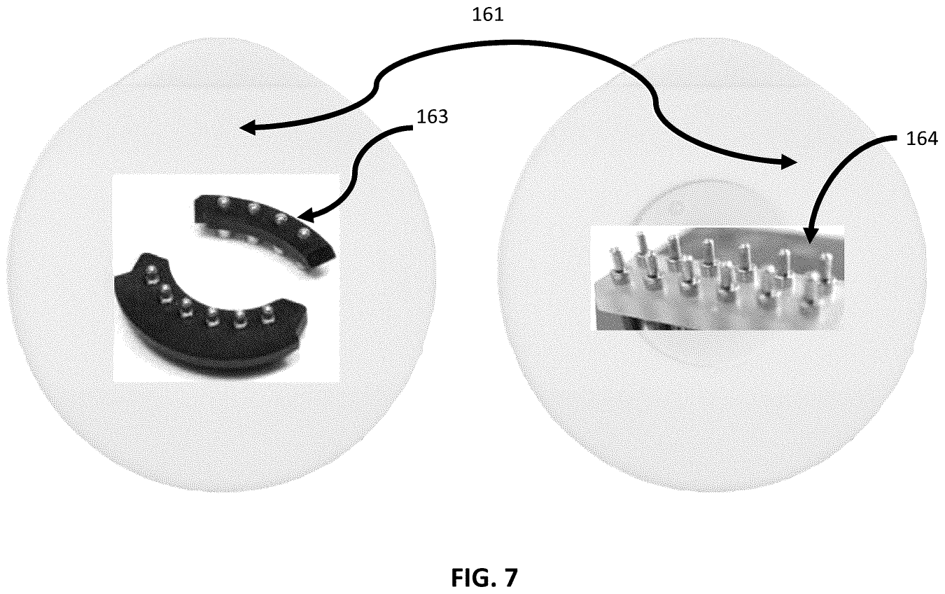

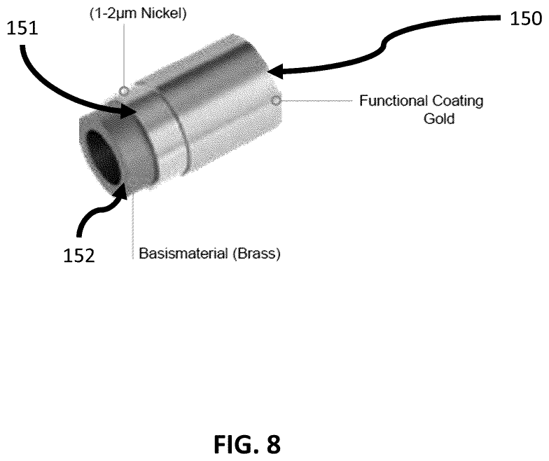

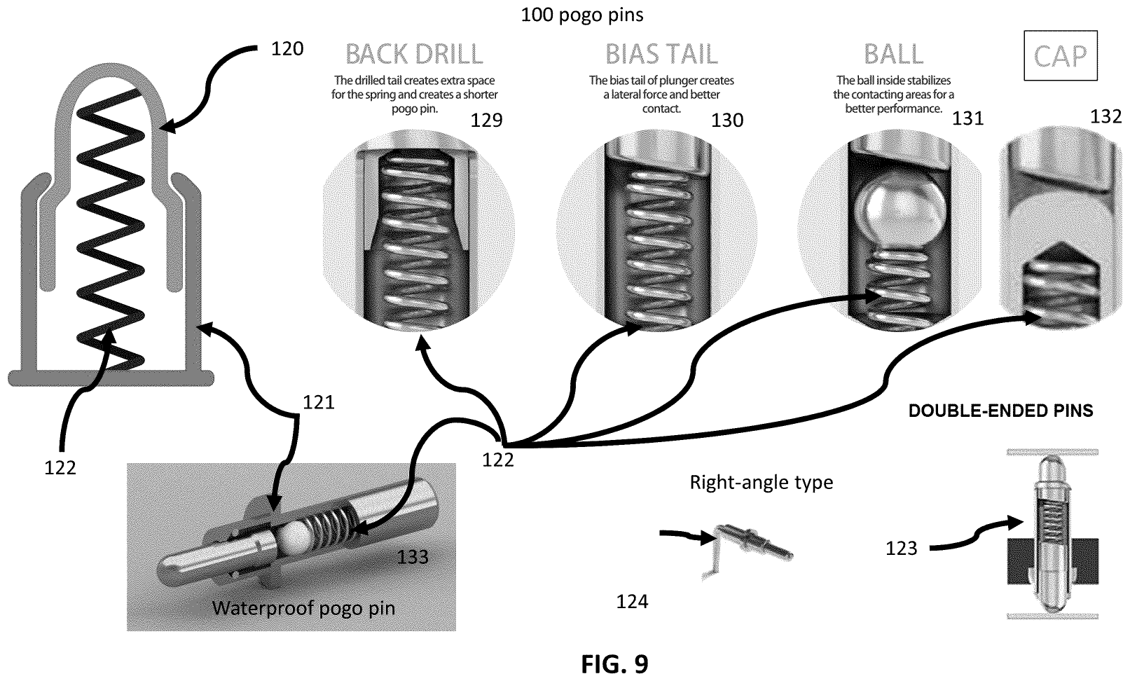

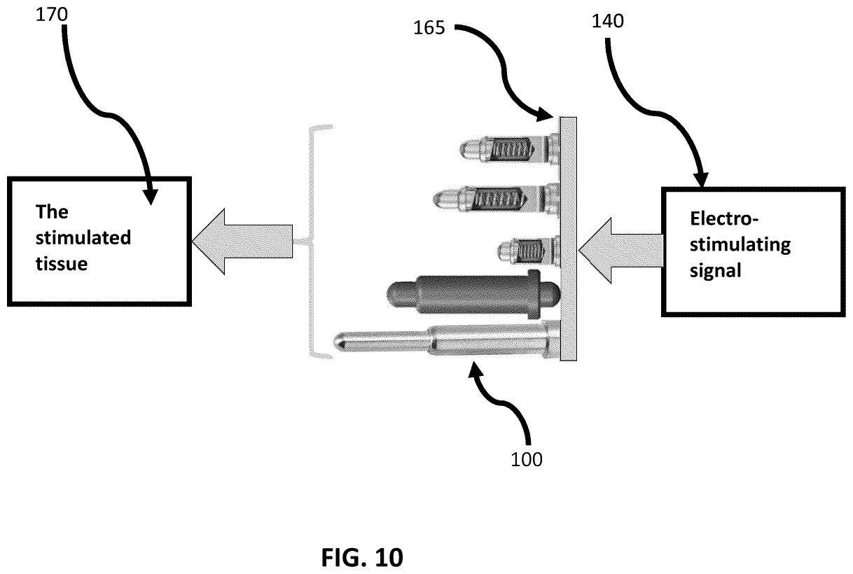

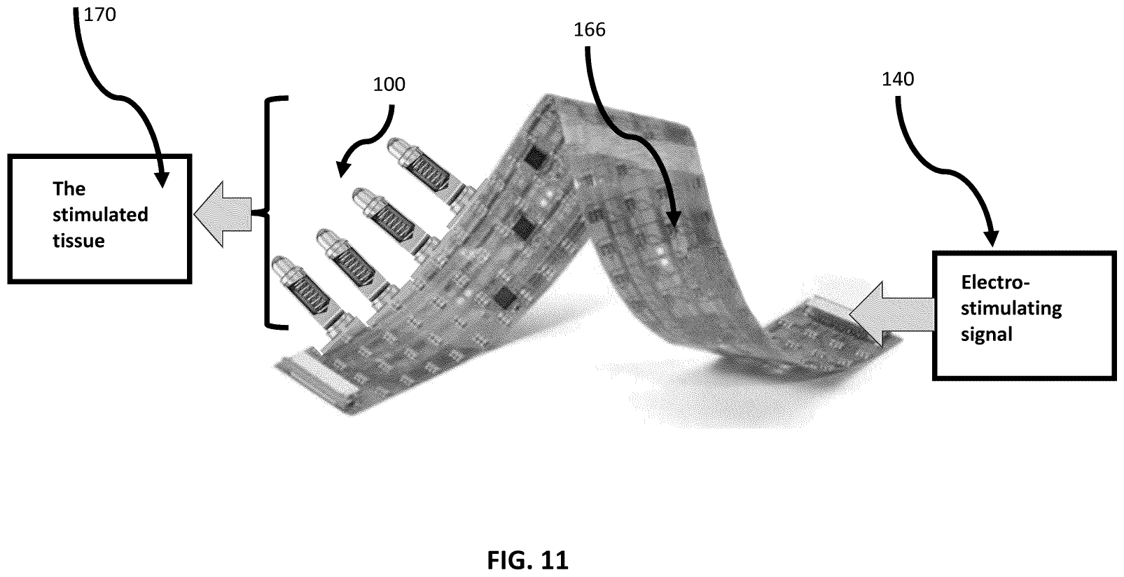

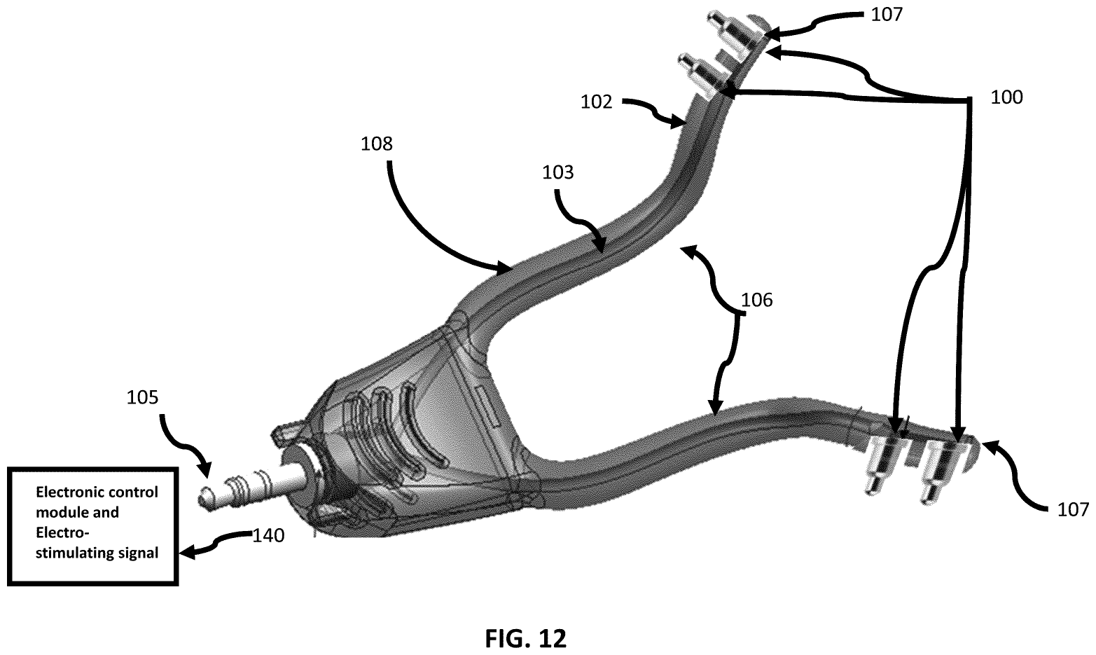

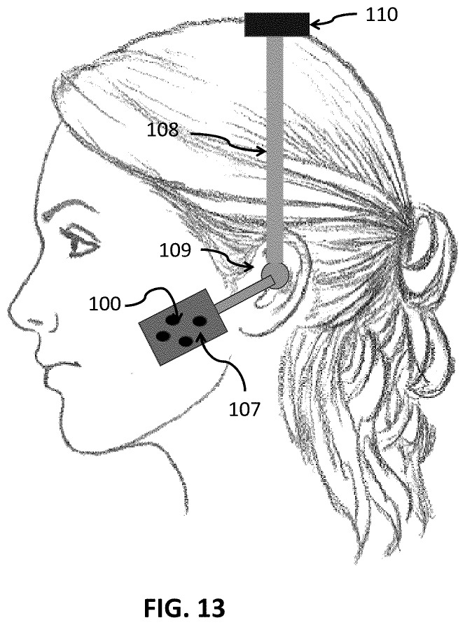

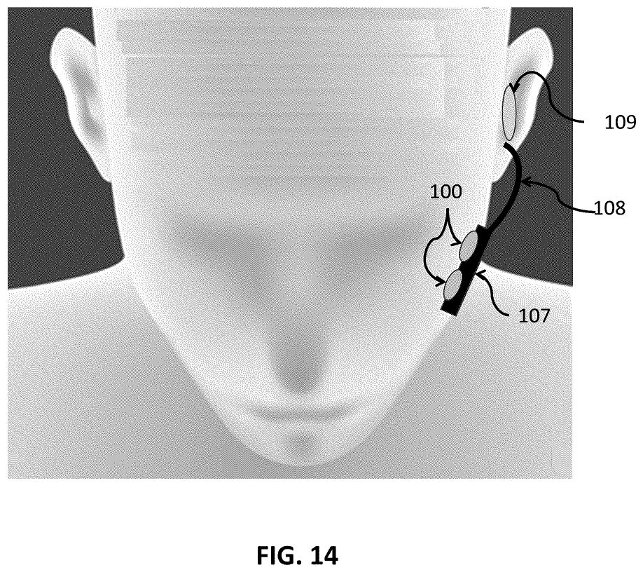

The electro-stimulating electrode can be configured with a single pogo pin (refer to FIG. 6), two pogo pins as demonstrated in the saliva electro-stimulator (see FIG. 12), or with multiple pogo pins arranged in any desired pattern that is deemed most effective. Examples of various configurations can be observed in FIG. 7, among others.

In an additional embodiment of the current invention, the electro-stimulator comprises an array or plurality of pogo pins. These pogo pins are interconnected in various setups, illustrated in FIG. 7 as an example. Leveraging the spring mechanism within each pogo pin and the array structure of the electrode, this design yields a flexible electro-stimulator capable of adapting to the dynamic contours of the skin while maintaining optimal contact. This ensures reliable contact, ensuring that at least one or more pins establish contact with the body's skin to deliver electro-stimulating signals.

Each pin operates as an independent electro-stimulator, and any number of pogo pins can be grouped into a single electro-stimulator, adopting diverse physical arrangements and combinations within the stimulating array. Moreover, each pogo pins or a group of pogo pins can be linked to a distinct electro-stimulator or signal generator.

The signal generator is typically controlled by a microprocessor, microcontroller, computer, or ASIC (Application-Specific Integrated Circuit) (110), allowing for the customization of stimulating patterns with a range of currents and voltages to suit specific requirements.

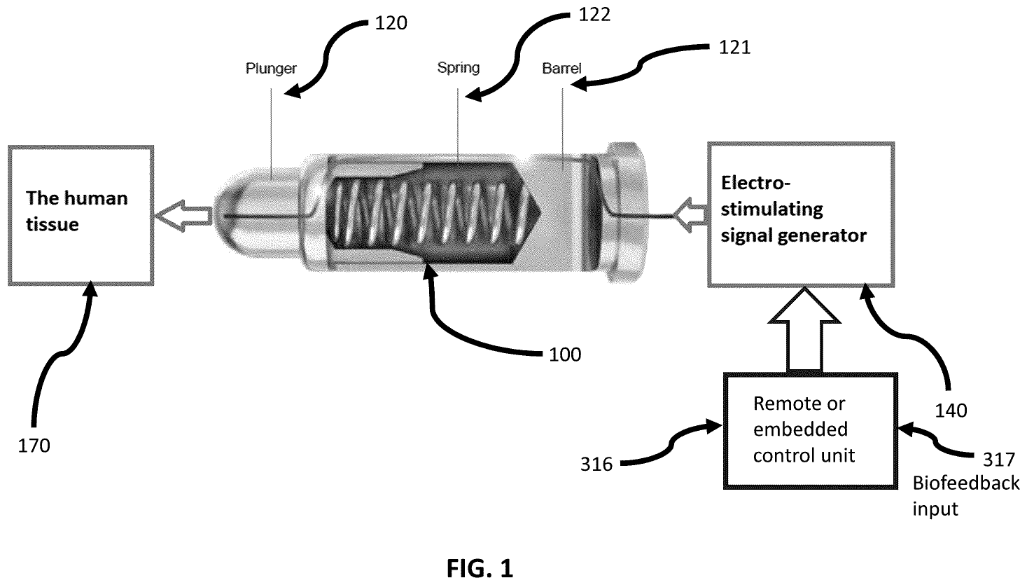

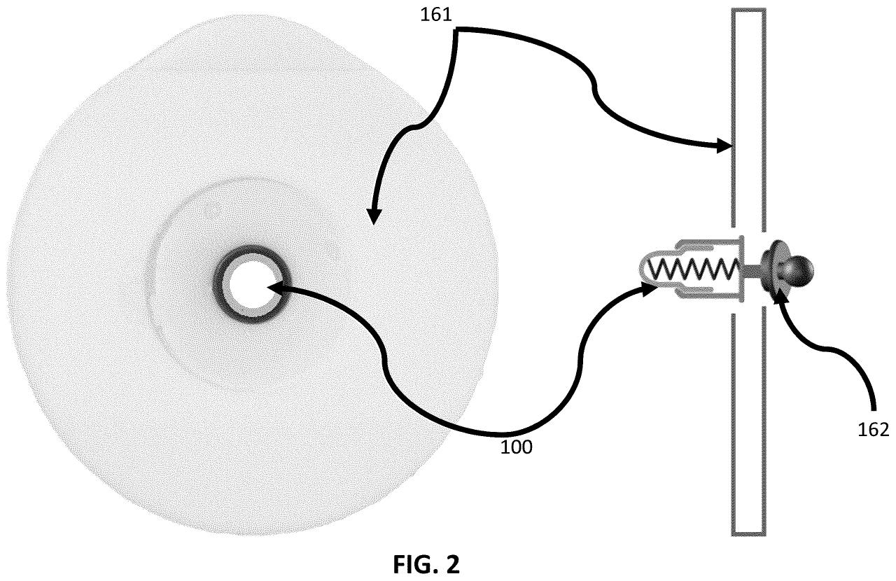

If each pogo pin is linked to an individual signal generator, each pin functions as an independent stimulator, as illustrated in FIG. 1. Conversely, when several pogo pins are connected to a single electro-stimulator or signal generator, each transmits an identical stimulating signal. In the event that one or more pogo pins are temporarily disconnected, the stimulating signal persists, as the remaining pins continue to transmit the signal.

Due to the distinctive spring-based structure of the pogo pins, they can be arranged in an array with the base (the connecting plane or plate) exhibiting rigidity, such as a rigid Printed Circuit Board (PCB) like FR4, or flexibility, such as a flexible PCB like Single-Sided PCBs, Double-Sided PCBs, Multilayer PCBs, and similar options. The momentary height of each pogo pin is determined by the inner spring, allowing them to conform to the tissue's contour. Placing the pogo pins on a Flex PCB, Rigid-Flex PCB, or similar configurations further enhances the overall flexibility of the electro-stimulator, ensuring its adaptability to the contours of the skin or the body tissue to which it is affixed.

Pogo pins provide higher current density VS. patches due to their small point of contact with the tissue. High current density is more effective in triggering evoked potential in nerves.

An additional innovation in the current embodiment involves distributing pressure across multiple pogo pins, leveraging their embedded springs. This design is well-suited for prolonged use of electro-stimulation while mitigating the risk of pressure ulcers, a challenge arising from the combination of prolonged pressure and time, which can result in tissue damage. Patient reactions to this combination can vary widely, and often, the underlying damage is severe by the time a surface wound becomes visible.

Furthermore, extended adherence of electro-stimulators to the skin can lead to irritation at the points of contact and perspiration underneath, impacting the contact stability in fixed pins. In contrast, the use of pogo pins proves more robust in such scenarios. The gold plating of the pogo pins, or the application of another noble metal coating, serves to prevent oxidation and maintain conductivity, preserving optimal performance-crucial considerations when electro-stimulation is employed over an extended duration.

Moreover, the inherent nature and biocompatibility of pogo pins contribute to minimizing degradation over time, reducing irritation, and mitigating sensitization effects on the stimulated tissue. This becomes particularly significant when electro-stimulation is applied for an extended period.

In an additional embodiment of the present invention, each electro-stimulator is not only constituted by an array of pogo pins arranged in a customizable format but also incorporates a diverse range of pogo pins heads. This feature facilitates tailoring specific combinations to address unique needs, such as accommodating dry skin, thick or hairy skin, and sweating skin, among others. This adaptability eliminates the requirement for skin preparation, messy gels, or irritating adhesives during stimulation.

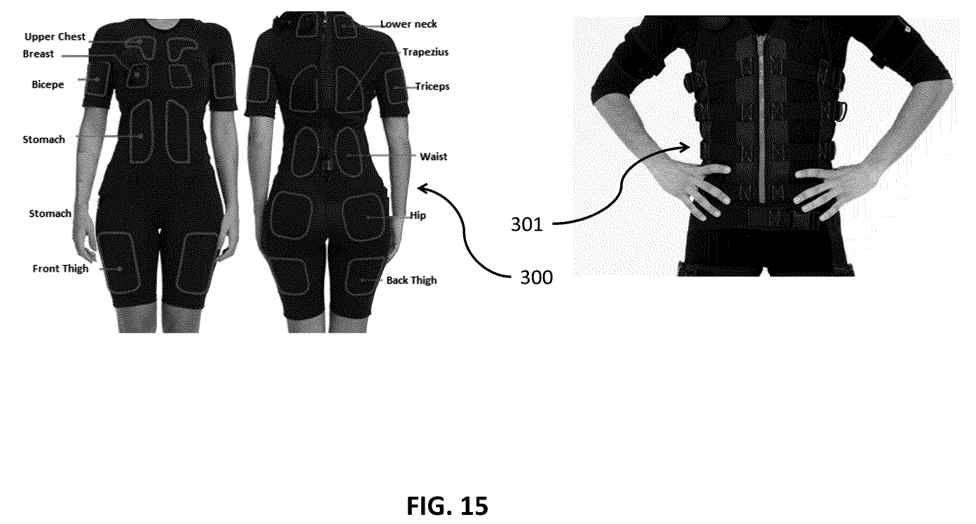

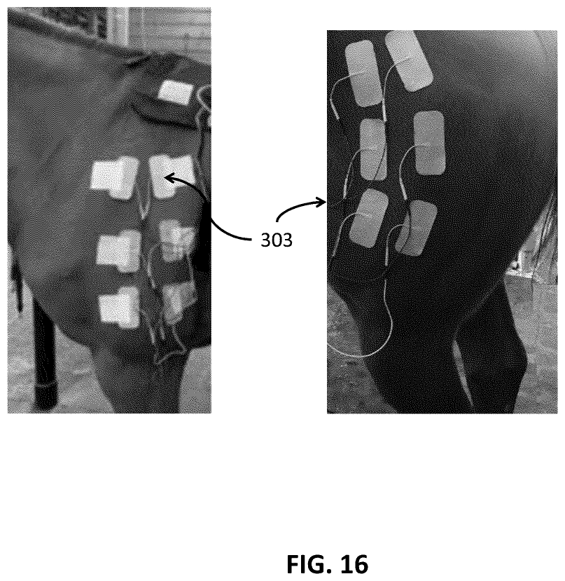

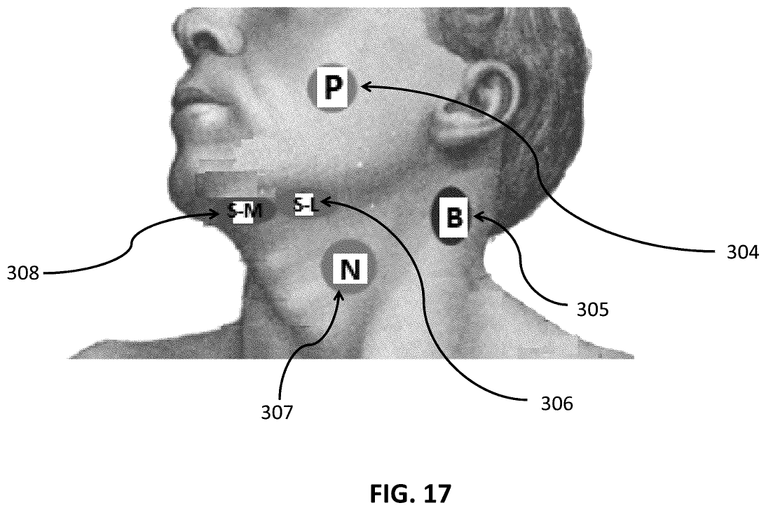

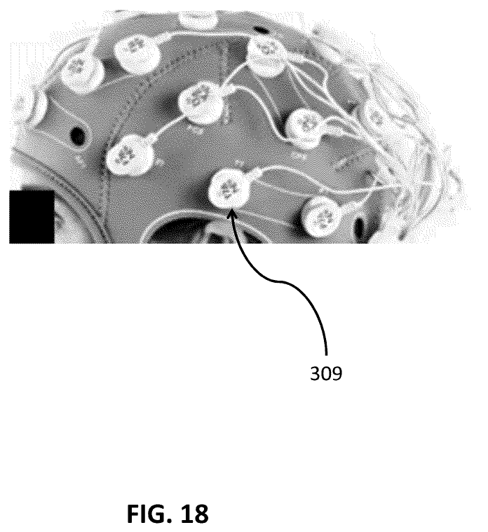

Another embodiment centers on wearable technologies designed for continuous electro-stimulation without the need for manual adjustments. See FIG. 15. Various wearable electro-stimulators, including those based on pogo pins, textile-based models (300), knitted integrated electro-stimulators, and fashionable planar circuit boards, find application in diverse electro-stimulation scenarios. A notable implementation in the present invention involves integrating an array of pogo pins into a wearable suit, such as on the upper torso (301) providing the advantage of electro-stimulation even during motion without causing tissue damage or compromising contact.

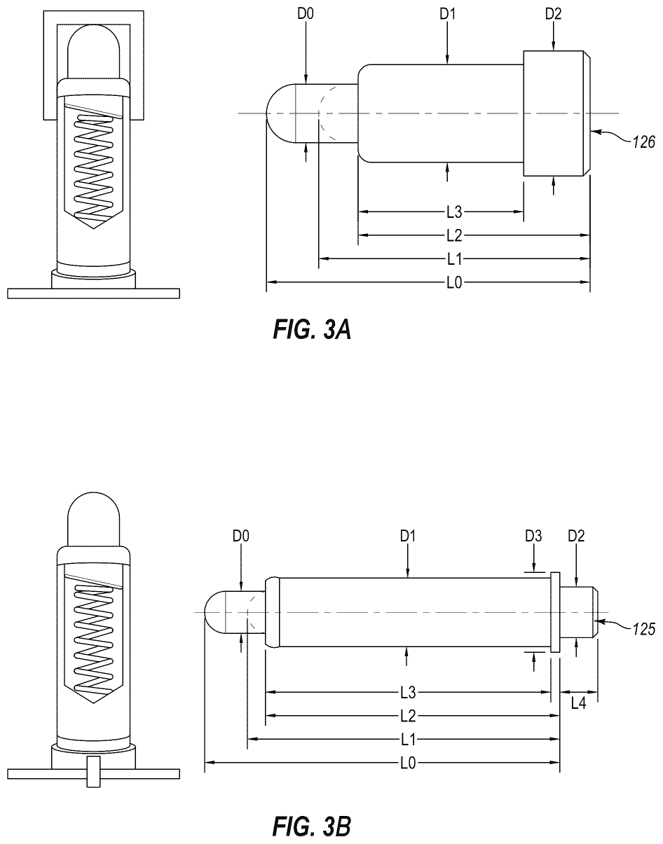

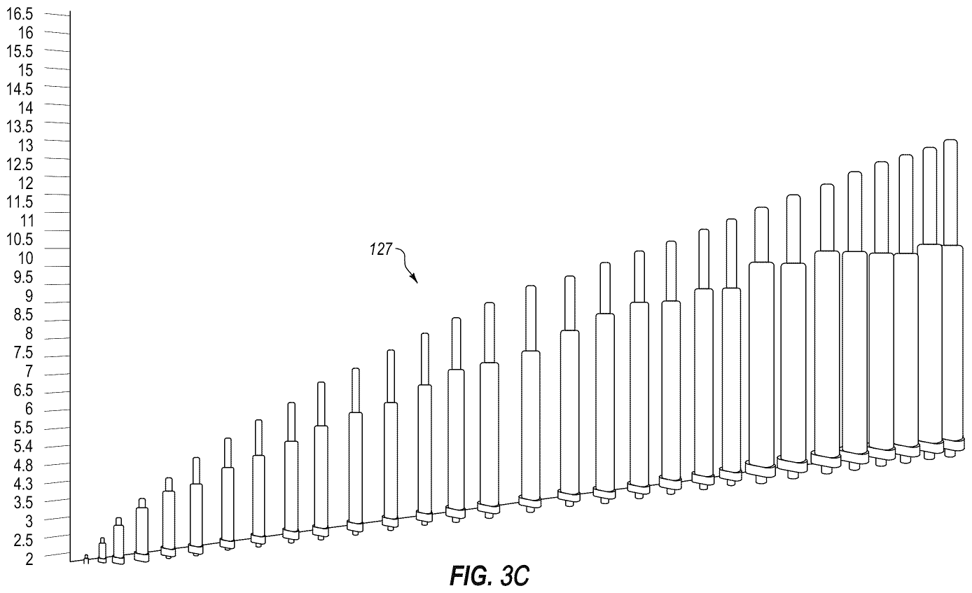

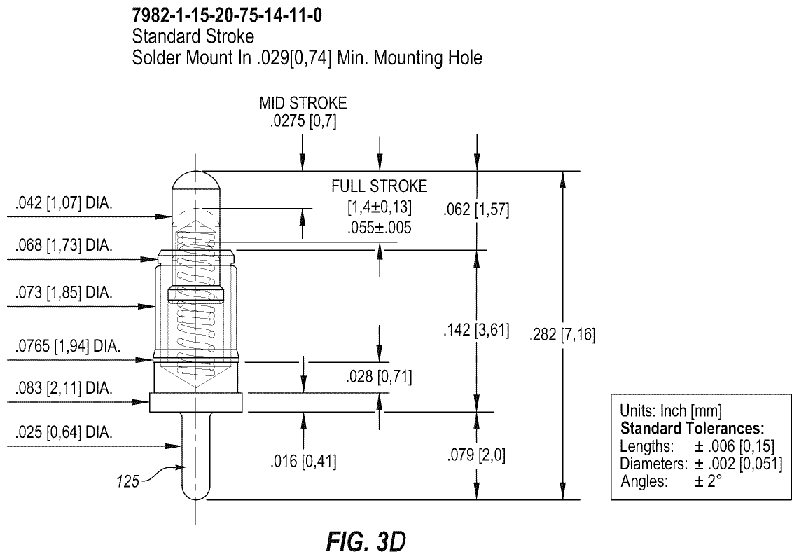

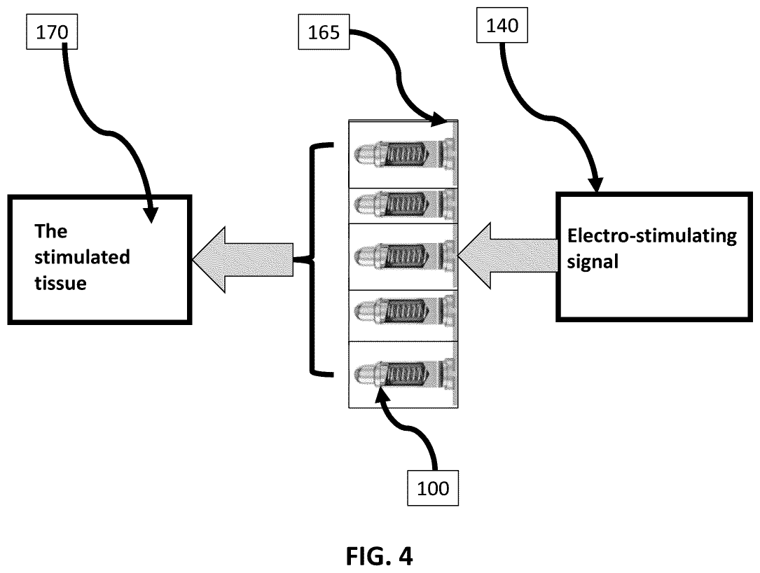

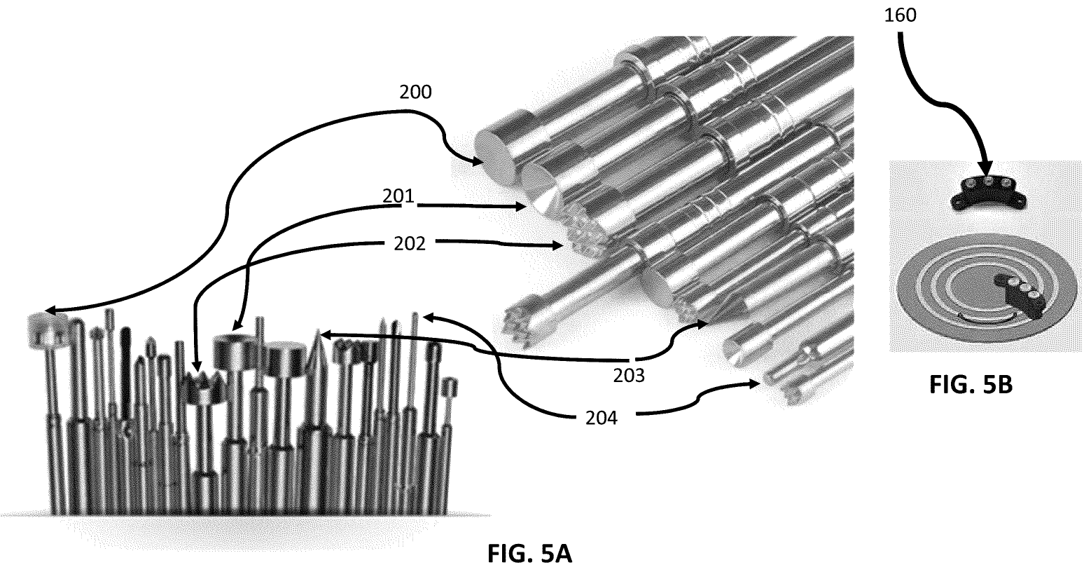

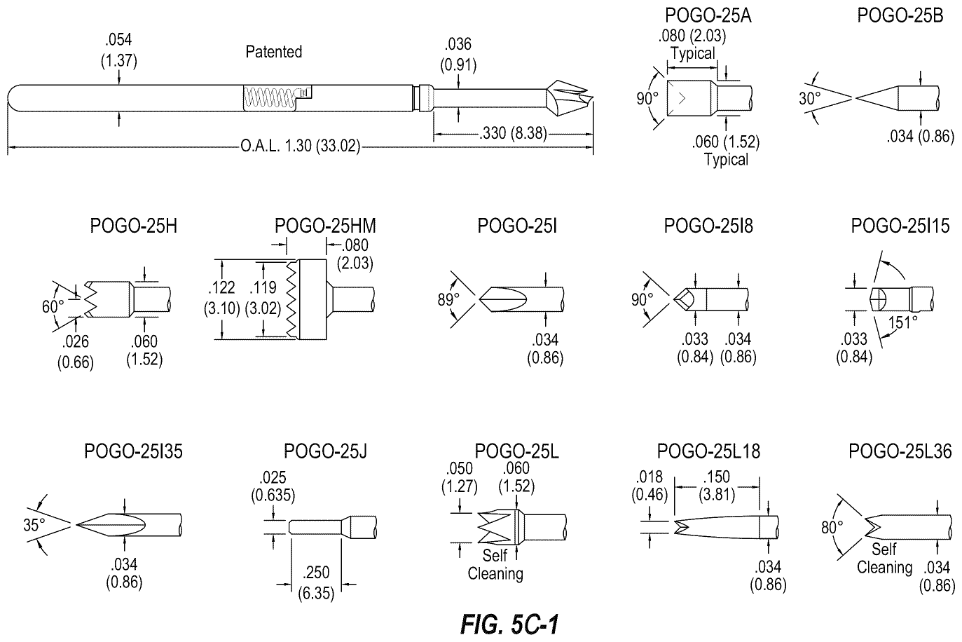

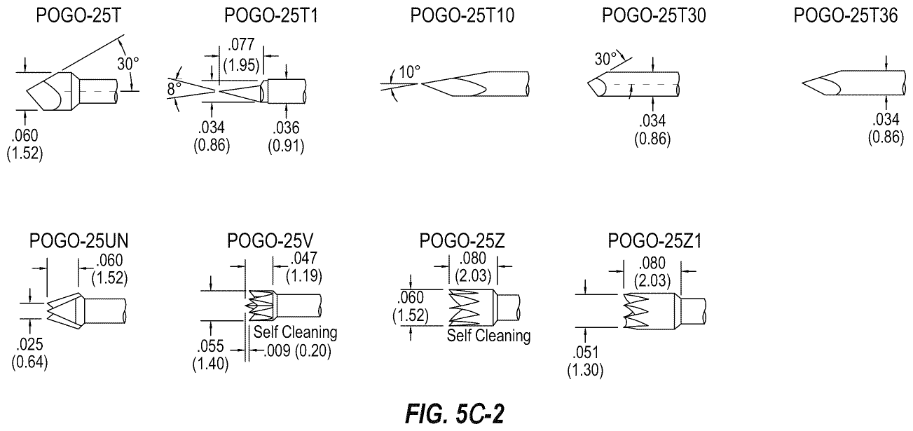

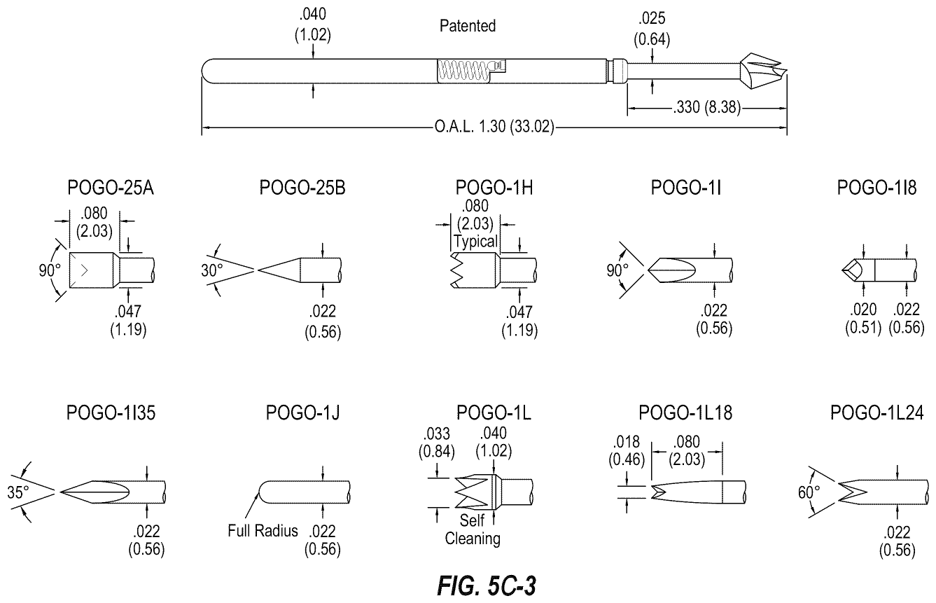

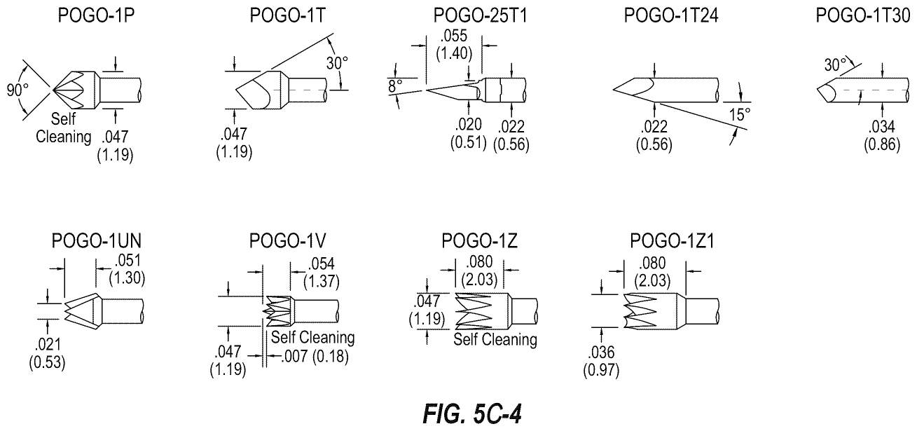

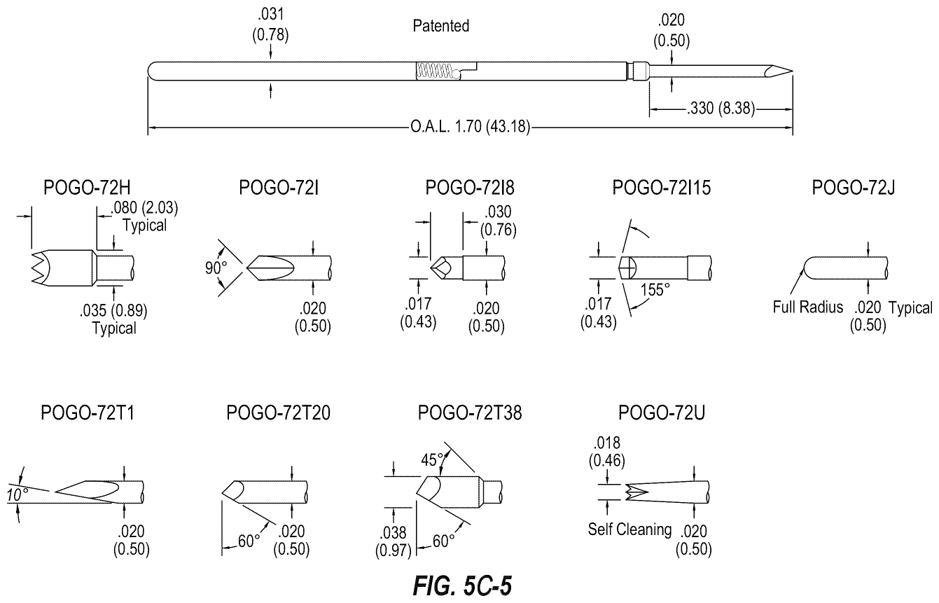

The versatility of pogo pins is underscored by their availability in different lengths (127), inner structures, current specifications, stroke movements (depicted in FIG. 3d, showcasing the displacement of the pogo pins edge due to spring compression), and soldering options (through hole 125 or SMD 126). This diverse range facilitates the selection of the most suitable pogo pins based on specific requirements and the location of the body where the wearable device is positioned.

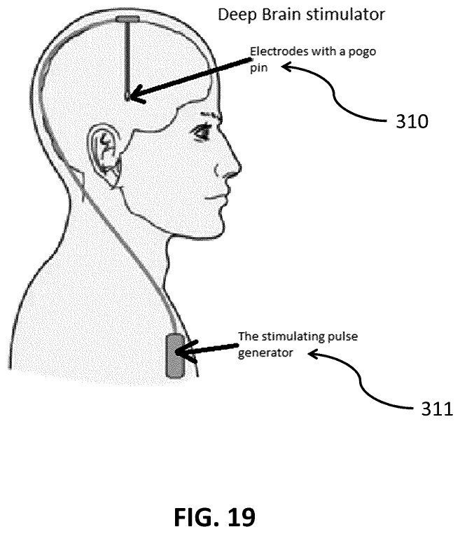

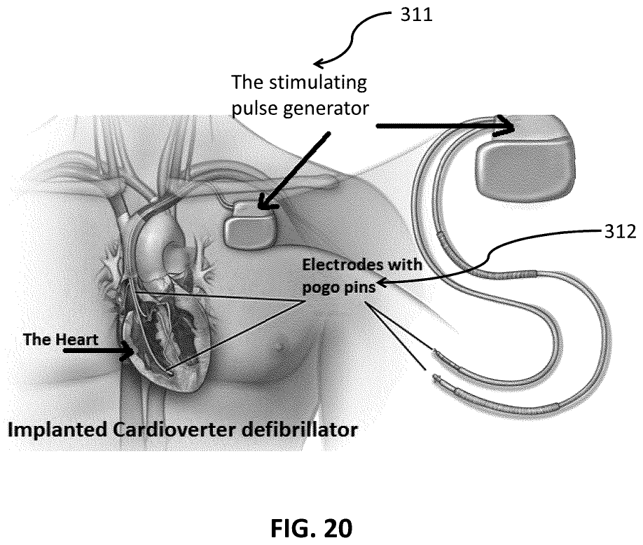

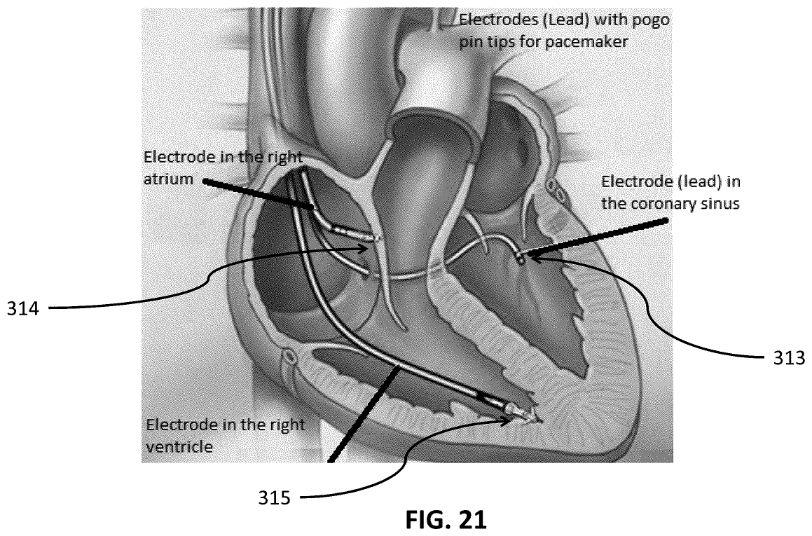

Another embodiment of the current invention uses pogo pins in an implanted electro stimulator such as a deep brain stimulator (DBS) FIG. 19, defibrillator FIG. 20, pacemaker FIG. 21, and the like, where the pogo pins contact the tissue and adjust itself to the changes in the tissue, such as due to the heart beats. Due to its unique structure, electrodes with pogo-pin at the tip can now be placed at other locations in the heart, where the movement of the tissue inhibits placing fixed-tip electrodes.

In an additional embodiment, the pogo pins are manufactured with non-magnetic materials. Non-magnetic pogo pins can fit many medical device applications, mainly in which the device may be subject to a strong electromagnetic field, like in MRI.

In an additional embodiment of the current invention, within the array of pogo pins, each pin can be connected to the signal generator through a resistor, capacitor, coil, or other active ICs. This arrangement ensures that the signal emitted from each pogo pin is deliberately shifted in time, phase, amplitude, current, or shape in comparison to neighboring pins, thereby generating a complex and varied stimulation pattern.

In an additional embodiment of the current invention, within the array of pogo pins, where the stimulating parameters and the position of the stimulating modules can be set by the user according to his/her biofeedback (317). The biofeedback can be in the form of placing the stimulating electrodes in a preferred location on the tissue such more saliva is secreted, pain reduction is better, or by setting the stimulating parameters to achieve a better effect of the electrostimulation. The parameters can be changing the stimulating time, duration, repetition, voltage, current, pulse duration, pulse frequency, pulse train frequency, and pulse shape (Rectangular, biphasic, single phase, sawtooth, and the like). The setting is performed via the embedded control options or via a remote-control unit (316), such as a mobile application that connects the mobile device to the stimulating signals module via Bluetooth, Zig-Bee, LBT, Wi-Fi, and the like.

### Examples

### An Example of TENS-Like Devices

This illustration focuses on Transcutaneous Electrical Nerve Stimulation (TENS or TNS), a therapeutic technique that utilizes electric current generated by a device to stimulate nerves. TENS encompasses the full spectrum of transcutaneously applied currents employed for nerve excitation, although the term is commonly employed with a more specific context, particularly to characterize the pulses generated by portable stimulators designed to alleviate pain. Typically, the unit is affixed to the skin using two or more electrodes, often in the form of conductive gel pads. A standard battery-operated TENS unit provides the capability to modulate pulse width, frequency, and intensity.

### An Example in Sport and Lifestyle

Electrostimulation in fitness, allows the trainee to achieve considerable results in the physical shape and wellbeing in a much shorter timeframe than conventional fitness workouts. Using Pogo pins as the electrodes, allows unlimited movement of the trainee, with very limited detachment of the electrodes from the tissue.

### Example of the Use in Veterinary

A nine-year-old polo pony sustained an injury when struck on the shoulder by a polo ball, resulting in damage to the suprascapular nerve and subsequent muscle atrophy in the supraspinatus and infraspinatus muscles. Electro-stimulation electrodes, utilizing a pogo-based system, were applied to stimulate neural firing and induce muscle contractions, aiming to prevent further disuse of the affected muscles. Initially, the goal was to alleviate pain through the use of anti-inflammatories, followed by efforts to enhance muscle strength, ultimately facilitating the rehabilitation of normal gait patterns.

### Example of Non-Magnetic Pogo Pins.

Pogo pins can be manufactured using non-ferro magnetic materials. Usually, the spring is made of stainless steel, but in non-magnetic pogo pins, it can be replaced with other materials such as Nitinol, Aluminum, Brass, Polymers with spring properties such as helical carbon-phenolic-based polymer, Polyacetal (POM) springs that made from the thermoplastic polymer polyoxymethylene. Using non-magnetic pogo pins in stimulating electrodes allows exposing the users to MRI tests, as an example.

### Example of Using Pogo Pins Electrodes in Implantable Devices

Implantable stimulating devices such as Deep Brain Stimulators DBS (used mainly by Parkinson's patients), Cardiac defibrillators for patients with cardiac arrest issues, and Pacemaker for cardiac patients with heart arrhythmia issues, use fixed electrodes, usually made of platinum. Pogo pins, with their properties, as disclosed in this patent, can replace the existing electrodes and provide better contact with the tissues, mainly, with tissues with continuous movement, such as the heart.

### Electro-Stimulation Market

Electrical stimulation devices play a major role in advanced healthcare. The global electrical stimulation devices market size was valued at US$3,956.0 million in 2017 and is expected to exhibit a CAGR of 4.7% over the forecast period (2018-2026).

Increasing incidences of spinal injuries are expected to propel the growth of the electrical stimulation devices market. Electrical stimulation devices are used in preoperative and postoperative pain management in patients with spinal injuries. A high incidence of spinal injuries caused due to accidents falls or violence is expected to propel the demand for spinal surgeries, which in turn is expected to drive the market growth. According to the World Health Organization (WHO) factsheet in 2018, annually there are around 37.3 million falls, that require medical attention, whereas about 646,000 individuals die each year, from falls globally. Moreover, increasing incidences of neurological disorders such as Alzheimer's disease and trauma associated with spinal injuries are expected to propel the growth of the electrical stimulation devices market over the forecast period. For instance, in August 2012, the National Center for Biotechnology Information (NCBI) stated Alzheimer's disease was the most frequent cause of dementia in Western societies and estimated around 5.5 million cases of the disease in the U.S., and 24 million globally.

The increasing prevalence of chronic diseases in North America is expected to bolster the market growth. North America holds a dominant position in the global electrical stimulation devices market, owing to a high prevalence of chronic diseases. According to the National Center for Biotechnology Information (NCBI), in March 2018, an estimated 45%, or around 33 million Americans were diagnosed with at least one chronic disease. Moreover, the rise in the geriatric population, who are highly susceptible to pain, is expected to bolster market growth over the forecast period. According to American Psychological Association, 2014 report, around 40 million people over the age of 65 years account for 14% of the total population, and around 60% to 75% of people over the age of 65 years are diagnosed with persistent pain.

1. A system for electro-stimulation of biological tissues, like nerves, muscles, and skin, utilizing pogo pins to contact the tissues, the system comprises;
a) stimulating signal generators generating a variety of electrical pulses aimed to stimulate the nerves, muscles, tissues, or skin;
b) a control module that controls the parameters of the electro-stimulating signals via an embedded application or a remote application;
c) connecting cables that link to one or more pogo-based electrodes to the stimulating signal generator;
d) one or more pogo pins arranged in various configurations that contact the tissues with the plunger side of the pogo pin and receive the stimulating signal at the barrel side of the pogo pin.

2. The system of claim 1, wherein the electrodes with the pogo pins are used for treating dry mouth using electrical stimulation, comprises:
a. One or more elastic arms for positioning and securely holding one or more stimulating electrodes;
b. One or more stimulating modules with electrodes made of pogo pin contacting the user's tissue, next to the lingual nerve or the auriculotemporal nerve comprising one or more plates, each featuring one or more pairs of pogo-pin electrodes.

3. The system of claim 1, wherein the electrodes with the pogo pins are used for cosmetic treatment such as eye lifting, anti-wrinkle treatments, facial care massage, eye and face massagers, skin smoothing, anti-aging solutions, skin tightening, skin lifting, skin rejuvenation, and similar cosmetic procedures, comprises;
a. array of electro-stimulators based on pogo-pins placed on the tissues that should be stimulated where the plunger side of pogo pins is facing and touching the tissue that needs the cosmetic treatment and the barrel side of the pogo-pin is connected to the stimulating-signals generators.

4. The system of claim 1, wherein the electrodes with the pogo pins are used for a sport where electrical impulses to various muscle groups, prompt involuntary contractions of a greater number of muscle fibers during training, leading to an elevated intensity of effort, comprises;
a) array of electro-stimulators based on pogo pins, where the plunger-side is facing and touching the tissue that is close to the various muscle groups that should be stimulated, and the barrel side of the pogo pin is connected to the stimulating-signals generators.

5. The system of claim 1, wherein the electrodes with the pogo pins are used for health therapeutic purposes for alleviating pain, managing preoperative and postoperative pain in patients with spinal injuries, improving blood circulation, facilitating physiotherapy and rehabilitation, treating dry eyes, aiding in stroke rehabilitation, increasing saliva production, managing incontinence, and supporting precision spinal cord stimulator, comprises;
a) array of electro-stimulators based on pogo-pins, where the plunger-side is facing and touching the tissues that are close to the organs that need treatment, and the barrel side of the pogo-pin is connected to the stimulating-signals generators.

6. The system of claim 1, wherein the electrodes with the pogo pins are used for implanted devices for therapeutic purposes, Deep Brain Stimulation, pacemaker, and defibrillator, comprises;
a) electro-stimulator based on pogo-pins, where the plunger-side is facing and touching the tissue that is close to the organs that need the stimulation, and the barrel side of the pogo-pin is connected to the stimulating-signals generators.

7. The system of claim 1, wherein the electrodes with the pogo pins are used for wearable electro-stimulators, textile-based models, knitted integrated electro-stimulators, and fashionable planar circuit boards, comprises;
a) an array of electro-stimulators based on pogo-pins, where the plunger-side is facing and touching the tissue that is close to the organs that need the stimulation, and the barrel side of the pogo-pin is connected to the stimulating-signals generators.

8) The system of claim 1, wherein the electrodes with the pogo pins are used for treating injuries of animals like horses, comprises;
a) an array of electro-stimulators based on pogo-pins, where the plunger-side is facing and touching the tissue that is close to the organs that need the stimulation, and the barrel side of the pogo-pin is connected to the stimulating-signals generators.

9) A method of electro-stimulation using pogo pins as the stimulating electrodes, where the plunger side of the pogo pin touches the tissue-skin, nerve, and muscle, and the barrel side is connected to the stimulating signal generator.

10) A method of electro-stimulation using pogo pins as the stimulating electrodes, wherein the electronic stimulating parameters and the position of the stimulating modules can be user-customized based on biofeedback.
